(19) United States
(12) Patent Application Publication
(10) Pub. No.: US 2006/0286569 A1
(43) Pub. Date: Dec. 21, 2006

(54) Method, apparatus, and system for authentication using labels containing nucleotide sequences

(76) Inventors: Yuval A. Bar-Or, Sunnyvale, CA (US); Paul O. Scheibe, Arroyo Grande, CA (US); Kevin W. Plaxco, Santa Barbara, CA (US); Arthur J. Thomas, Oxford (GB)

Correspondence Address: SCHNECK & SCHNECK, P.O. BOX 2-E, SAN JOSE, CA, 95109-0005, US

(21) Appl. No.: 11/370,531

(22) Filed: Mar. 8, 2006

Related U.S. Application Data
(60) Provisional application No. 60/660,758, filed on Mar. 10, 2005.

(51) Int. Cl. C12Q 1/68; G06F 19/00

(52) U.S. Cl. 435/6; 702/20; 380/59

(57) ABSTRACT

A method, label, and labeling system for labeling and authenticating an item are presented. At least one of a number of known nucleotide sequences associated with a predetermined amount of information is used as a label to be associated with an item. The label is then read with a reagentless sensor to detect the nucleotide sequence(s). The detected nucleotide sequence(s) is then associated with the appropriate information. The item is authenticated if the sensor detects the expected nucleotide sequence(s). The information in the DNA label may also be passed through a hash function or encrypted to further enhance security. The labels may also incorporate known non-natural nucleic acid analog sequences rather than nucleotide sequences, and a reader that reads known non-natural nucleic acid analog sequences may be employed.

### CROSS-REFERENCE TO RELATED APPLICATION

This application claims priority from U.S. provisional application No. 60/660,758, filed Mar. 10, 2005.

### TECHNICAL FIELD

This invention concerns authentication of items, particularly those items having a label containing nucleotide sequences.

### BACKGROUND OF THE INVENTION

As is well known, deoxyribonucleic acid (“DNA”) is an organic acid found in the nucleus and mitochondria of a cell. DNA consists of two long chains of nucleotide bases that are twisted around each other in a structure called a double helix. There are four bases in DNA (cytosine, guanine, adenine, and thymine); each of the bases in each chain forms complementary base pair with a base in the other chain (for instance, guanosine forms a base pair with cytosine, adenine forms a base pair with thymine). One chain of the double helix is the complement of the other chain; two single-strand DNA (“ssDNA”) molecules bind to form a double helix in a process known as hybridization if the two single-strand sequences are complements of each other and conditions are otherwise conducive to hybridization.

DNA is stable in many environments and has enormous coding capacity—a DNA sequence of as few as 17 bases has over 17 billion unique sequence combinations. Therefore, even short sequences of DNA can be highly effective when used in labels to identify, authenticate, and verify an item associated with the label. This has been discussed in U.S. Pat. Nos. 5,139,812 and 6,312,911.

A rapid, inexpensive, and sequence-specific method for detecting nanogram quantities of short ssDNA sequences has been developed by C. Fan, K. W. Plaxco, and A. J. Heeger. Their method has been described in U.S. Patent Application Publication No. 20040191801 (Ser. No. 10/678,760), “Reagentless, Reusable Bioelectronic Detectors and Their Use as Authentication Devices,” filed Oct. 3, 2003, the contents of which are herein incorporated by reference, U.S. Patent Application Publication No. 20050112605 (Ser. No. 10/810,333), “Reagentless, Reusable Bioelectronic Detectors and Their Use as Authentication Devices,” filed Mar. 25, 2004, the contents of which are herein incorporated by reference, and in a scientific paper “Electrochemical Interrogation of Conformation Changes as a Reagentless Method for the Sequence-specific Detection of DNA,” PNAS, vol. 100, no. 16, pp. 9134-9137 (Aug. 5, 2003), the contents of which are herein incorporated by reference. An electrochemical DNA (“E-DNA”) sensor is employed which consists of a gold electrode which is coated with loop-shaped DNA molecules (the DNA molecule has a ferrocene tag at the 5′ end and a thiol at the 3′ end, with five complementary bases at its 5′ and 3′ ends which bind to each other, forming the stem loop). The loop-shaped molecule's tail is held close to the surface of the gold electrode when the molecule is not bound to a complementary DNA (“cDNA”) sequence; when binding with a complementary DNA sequence occurs, the DNA molecule on the electrode undergoes a conformational change and assumes a “stretched” shape, where the tail is held further away from the electrode. The distance between the ferrocene tag and the electrode is significantly increased and a signal change is measured (the ferrocene tag produces an electric current when held close to the gold electrode but does not when it is held away from the electrode) using cyclic voltammetry, indicating that hybridization has occurred. The E-DNA sensor can detect binding of complementary DNA sequences without the use of exogenous reagents and without employing optics, light sources, or photodetectors. The E-DNA sensor may detect specific, short DNA sequences at concentrations as low as 150 picograms per milliliter. These short sequences may be detected without having to amplify the target DNA sequence, for instance via polymerase chain reaction (which also requires primer oligonucleotides). The E-DNA sensor may also be configured to detect several target DNA sequences. Other potential configurations of the sensor and preparation of the tag, loop-shaped molecule, complementary sequence, and label as well as descriptions of operation and experimental results, are described in the patent application and article incorporated by reference, above.

In FIG. 1, a stem loop oligonucleotide 12 possessing a terminal thiol and methylene blue (MB) tag 14 (here used instead of a ferrocene tag) is immobilized at a gold electrode 10. The E-DNA sensor detects the voltage 16 due to the MB tag 14 being relatively close the surface of the gold electrode 10. When hybridization occurs due to the presence of a complementary DNA sequence, the MB tag 14 on the resulting two-stranded molecule 18 is held further away from the electrode 10 than it was before hybridization and no signal 20 is detected. FIG. 2 shows the current measured at different concentrations of target DNA.

FIG. 3 shows how a DNA label may be used in an authentication process. As provided in paragraphs 109 and 111 of U.S. Patent Application Publication No. 20040191801, incorporated by reference, above, and paragraphs 116 and 118 of U.S. Patent Application Publication No. 20050112605, incorporated by reference above, 1 mL of a DNA solution (approximately 5 ng of target oligonucleotide sequence 5′-ACTGGCCGTCGTTTTAC-3′ (fully complementary to the oligonucleotides sequence on the E-DNA sensor) with 10,000-fold excess of non-cognate oligonucleotides sequence 5′-CGTATCATTGGACTGGC-3′ (a sequence unrelated to the probe or target sequence and used as masking DNA)) was added to a small circle (approximately 3 mm in diameter) printed on filter paper with a ball pen. After drying, the DNA microdot was cut from the paper and immersed in 20 mL salt water containing 10 mM phosphate buffer with pH 7.0 and 1 M NaCl for approximately 10 minutes. Two mL of the eluted solution was placed at the E-DNA electrode surface. After a thirty minute hybridization period, the AC voltage dropped by approximately forty percent. When a DNA microdot with only 50 mg of masking DNA was used, the E-DNA signal remained almost unchanged. In this process, pre-identified DNA sequence is placed on a label made of filter paper (other inert material, such as letter paper may be used) (block 100). In order to authenticate the item, the label is placed in a solution, such as a salt water solution, to elute the DNA. The E-DNA sensor is placed in the solution containing the label, and the E-DNA sensor, or reader, detects whether the target DNA sequence is present (block 102) (the E-DNA sensor has the complementary sequence of the pre-identified target DNA attached to the electrode; if the target DNA sequence is present, it would bind with the complementary sequence on the sensor's electrode and a signal indicates binding has taken place (here, the measured voltage drops)). If the target DNA sequence is present (block 104), the item with which the label is associated is authenticated (block 106). If the target DNA sequence is not detected (block 104), the item is not authenticated (block 108).

DNA labels may also be used in orally ingested or injectable drugs. As provided in paragraphs 112 and 113 of U.S. Patent Application Publication No. 20040191801, incorporated by reference, above, and paragraphs 116 and 118 of U.S. Patent Application Publication No. 20050112605, incorporated by reference above, Lipitor tablets (Pfizer) were ground into a powder and approximately 1 microliter of DNA (20 ng of target oligonucleotide sequence 5′-ACTGGCCGTCGTTTTAC-3′ (fully complementary to the oligonucleotides sequence on the E-DNA sensor) and 200 mg masking DNA) was added to the powder. After drying in the air, the powder was dispersed in 50 ml salt water and then filtered to obtain the supernatant. One mL of liquid Neupogen (Amgen) was mixed with 1 mL DNA (20 ng of target oligonucleotide sequence 5′-ACTGGCCGTCGTTTTAC-3′ (fully complementary to the oligonucleotides sequence on the E-DNA sensor) and 200 mg masking DNA), diluted into a 50 mL solution. Two mL of this solution was pipetted on the gold electrode surface of the E-DNA sensor. After a thirty minute hybridization period, the AC voltage dropped significantly for both the Lipitor and Neupogen samples, while significantly smaller AC voltage drops were observed in the control experiments (i.e., those experiments where no target oligonucleotide sequence was employed).

A label with a short ssDNA sequence which may be detected by the above-mentioned sensor may be used to identify and authenticate an item with which the label is associated. Given the selectivity of the E-DNA sensor, the ssDNA sequence may be incorporated with other DNA sequences for the purpose of “masking” the correct DNA sequence to add further security to the identification/authentication label. However, the security of such a label may be compromised, for instance, by switching labels and detectors or by an “adversary” who knows which DNA sequences are used for authentication and producing labels with the sequences and attaching them to, for instance, counterfeit items. Therefore, it would be desirable to provide a label and labeling system (along with corresponding methods) for identification, authentication, and verification purposes that offers greater security than currently-known approaches. It would also be useful to have a label and labeling system that could provide information in addition to authentication. In addition, a label and labeling system that would provide means for detecting any tampering with the label would be advantageous.

### SUMMARY OF THE INVENTION

Providing labels with DNA or other nucleotide sequences which encode information and may be combined with additional cryptographic methods offers an extremely secure identification and authentication method and system which may convey additional information about the item with which the label is associated.

In one embodiment of the invention, a method for labeling an item comprises using at least one of a number of known nucleotide sequences or known non-natural nucleic acid analog sequences associated with a predetermined amount of information as a label to be associated with the item, reading the label to detect the at least one known nucleotide sequence or known non-natural nucleic acid analog sequences, and authenticating the item if the read label contains the at least one identified nucleotide sequence or known non-natural nucleic acid analog sequences.

In another embodiment of the invention, a label for an item comprises information authenticating the item, wherein the information includes at least one known nucleotide sequence or at least one known non-natural nucleic acid analog sequences associated with a predetermined amount of data which may be detected with a reagentless sensor, the information authenticating or providing information about the item associated with the label.

### BRIEF DESCRIPTION OF THE DRAWINGS

FIG. 1 is a diagram showing how a prior art E-DNA sensor detects when hybridization has taken place.

FIG. 2 is a graph of results from a prior art E-DNA sensor showing current in the presence of complementary DNA at different concentrations.

FIG. 3 is a flowchart showing how a DNA label may be used in an authentication process in the prior art.

FIG. 4 is a flowchart showing how a DNA label may be encoded and read in accordance with the invention.

FIG. 5a is a flowchart showing how information to be encoded into a DNA label may be passed through a keyed hash function in accordance with the invention.

FIG. 5b is a flowchart showing how a DNA label encoded with information passed through a keyed hash function may be read to authenticate the item associated with the label in accordance with the invention.

FIG. 6a is a flowchart showing how information to be encoded into a DNA label may be encrypted in accordance with the invention.

FIG. 6b is a flowchart showing how a DNA label encoded with encrypted information may be read to authenticate the item associated with the label in accordance with the invention.

FIG. 7a is a flowchart showing how a DNA label may be employed with another product marking material in accordance with the invention.

FIG. 7b is a flowchart showing how a DNA label employed with another product marking material may be read to authenticate the item associated with the label in accordance with the invention.

FIG. 8a is a flowchart showing how a DNA label may be employed with another product marking material in accordance with the invention.

FIG. 8b is a flowchart showing how a DNA label employed with another product marking material may be read to authenticate the item associated with the label in accordance with the invention.

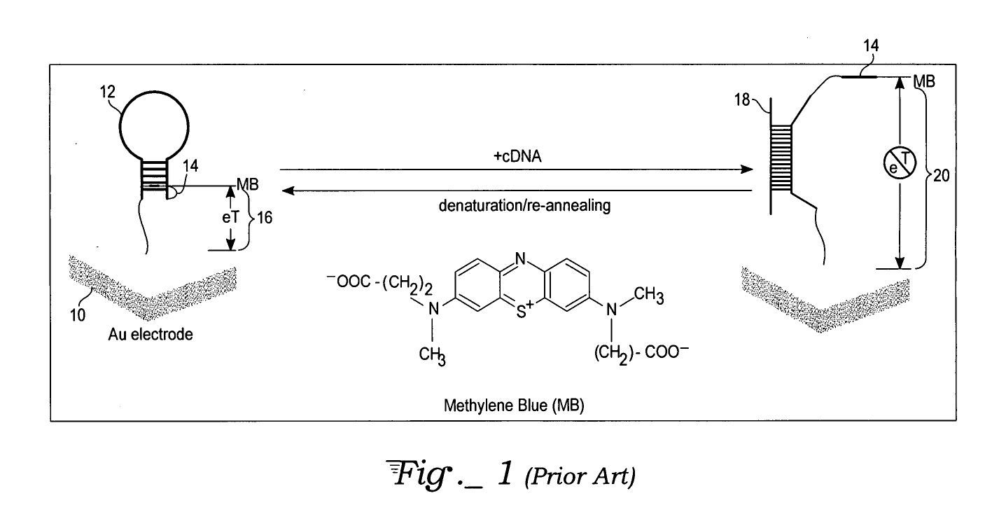

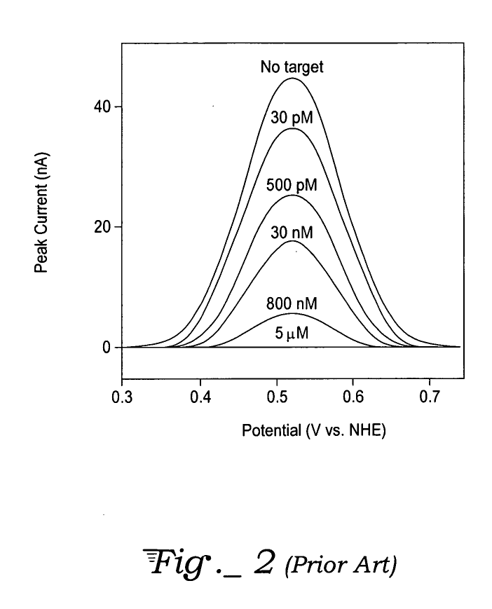

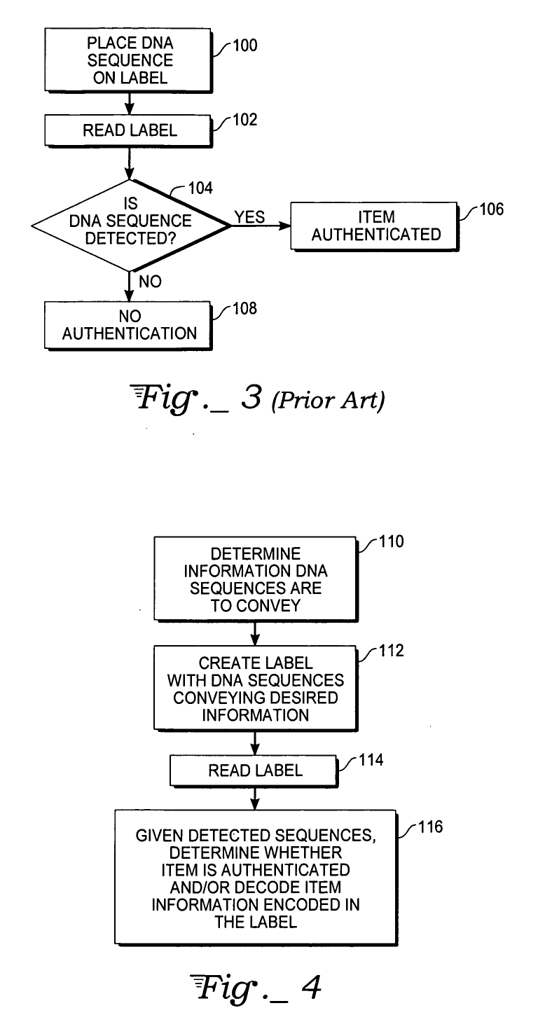

### DETAILED DESCRIPTION OF THE INVENTION

The following definitions for these terms are used throughout the application (unless otherwise noted):

DNA tag—a mix of oligonucleotides that provides a unique signal when appropriately interrogated.

DNA label—a self-contained authentication label using a DNA tag. The label may be associated with an item to be labeled, but is distinct from the item (though the label may be applied to the item). A label may be created, for instance, by placing the DNA tag on a surface (such as paper, plastic, or any appropriate substrate) which is then affixed to an item, placing the DNA tag directly on a surface of the item, or mixing the DNA tag in a liquid, or otherwise including the tag in the item. Labels may also be added to various media, including liquids, powders, solids, gels, gases, etc. They may be applied to any external surface or any internal surface of an item to be analyzed.

DNA reader—a device for interrogating a DNA label to determine its signal and make the association with a unique or known class of signals. In one embodiment, a reader comprises a sensor as described above in electrical connection with a computing device which receives the signals from the sensor and, using software, hardware, or a combination of software and hardware, interprets the signals, based on a predetermined association of signals with data and, where applicable, knowledge of the hash key used to encode the data in the label and encryption schemes used to encrypt the data in the label, to authenticate the labels and/or obtain information from the labels. (Other sensors may be employed in other embodiments.) The reader should be able to display results and/or transfer data to another device (either via a direct or network correction or downloading results to a removable storage media).

Label components—self-contained disjoint parts of a DNA label.

Component configuration—enumerated valid relative layout of parts or elements of a component.

Although DNA sequences, DNA tags, DNA labels, and DNA readers are mentioned throughout the application, it should be understood that any nucleotide sequence, as well as any non-natural nucleic acid analog sequence (including, but not limited to, peptide nucleic acid (“PNA”), threose-based nucleic acid (“TNA”), or L-deoxyribose, may be employed in this invention to create tags and labels. In addition, any reader, such as the sensor described above, capable of discerning nucleotide sequences, as well as non-natural nucleic acid analog sequences, may be employed. In one embodiment, DNA sequences which are 20 base pairs long are used; however, different embodiments may employ sequences of different length. (Security improves with the length of the sequence.)

Introducing uncertainty into the configuration of the label increases the security of the label and labeling system by making it more difficult to circumvent the authenticity of a label. As is known in information theory, entropy measures the uncertainty of a random variable. If the i-th component of a label has nᵢ configurations and the probability of the j-th configuration is pᵢⱼ, then the entropy of the component is:

$${H}_{i} = - {\sum }_{j = 1}^{{n}_{i}} {p}_{ij} log {p}_{ij}$$

and Hᵢ is in bits if base 2 is used for the logarithm. If the configuration of the i-th component is known, then Hᵢ=0 since

$${lim}_{p \to 0} p log p = 0.$$

Hᵢ=log nᵢ for a uniform distribution. If a sequence in a label component is 17 bases long, where one of four bases is equally likely at each position in the sequence and each base choice is a distinct configuration, the entropy is 34 bits. Additional uncertainty may be generated by introducing a random number, or key, that is kept secret.

In the present invention, information is conveyed by the presence, or absence, of one or more specific DNA sequences. By encoding a label with various DNA sequences, information about the item with which the label is associated may also be encoded in the label. The specific DNA sequence which is encoded in the label provides the information. For instance, in one embodiment, three bits of information may be represented by selecting one of eight different molecules to place on the label (in binary encoding, a bit of information may be a 1 or 0, i.e., a single bit can indicate 2 states, or 2 different pieces of information. If three bits of information are desired, any one of the eight molecules may be used to convey that information since 2³=8). Other embodiments may employ different numbers of sequences to encode the desired information. Using this approach, any information may be encoded in the label, including, but not limited to, serial number of an item, manufacture code of an item, date of manufacture of an item, date of expiration of an item, authentication information, etc.

More information may be encoded by placing DNA at specific locations in a geometric pattern. For instance, a particular sequence appearing at location “A” may encode one piece of information, and the same sequence appearing at location “B” may encode another piece of information. An array containing n possible DNA locations provides a maximum of n times the information contained in a single location. There are a number of ways to detect sequences at different locations. These include using an array of detectors, scanning with a single detector, or removing the material around each DNA location individually and determining the presence or absence and kind of DNA in a separate array of detectors or sequentially with a single detector. In one embodiment, a technician would remove each of the pattern “spots” containing a DNA sequence in some pre-defined order and introduce each spot to a reader to detect the DNA sequence. In other embodiments, an electronic steering mechanism may be employed to cause the DNA at each spot to be released from the label surface in a particular order. In another embodiment, the entire label would be removed and placed in some defined orientation onto an electrode array within the reader, with elution being highly localized. In yet another embodiment, an electrode array forms part of the label; the DNA sequences are placed on the electrodes and the array is removed and placed on a reader to detect the DNA sequences at different locations.

In FIG. 4, the information that DNA sequences convey is determined (block 110). For instance, sequence 1 may represent 000, sequence 2 may represent 001, etc. The label with the appropriate DNA sequences conveying the desired information is then created and associated with the item (for instance, the label may be affixed to the item or the item's packaging) (block 112). To authenticate the item and decode the information encoded on the label, the label is placed in solution with the E-DNA sensor, and the label is then “read” (block 114). Given the detected sequences, a determination is then made of whether the item has been authenticated and/or the information about the item which is encoded in the label is decoded (block 116).

Binding the components of a label together may also be accomplished through use of a message authentication code such as, in one embodiment, a key-dependent one-way hash function. A one-way hash function F(M), where M is the message has the following properties: h=F(M) is a fixed-length bit string that depends on M (h is the hash result); given M, it is easy to compute h=F(M); given h, it is hard compute M such that F(M)=h; given M, it is hard to find another message M′ such that F(M)=F(M′); and it is hard to find two random messages, M and M′, such that F(M)=F(M′). A key-dependent one-way hash function, e.g., F(K,F(K,M)), where K denotes a string used as a key and the comma indicates concatenation of two strings, may be formed. In other embodiments, other hash functions may be employed. Any known hash algorithm, such as MD-5 and SHA-1, may be employed in any of the embodiments. The key may be one of the label components (although it is advisable to not include it as part of the actual label). When appropriately bound together, the components form a nonmalleable DNA label.

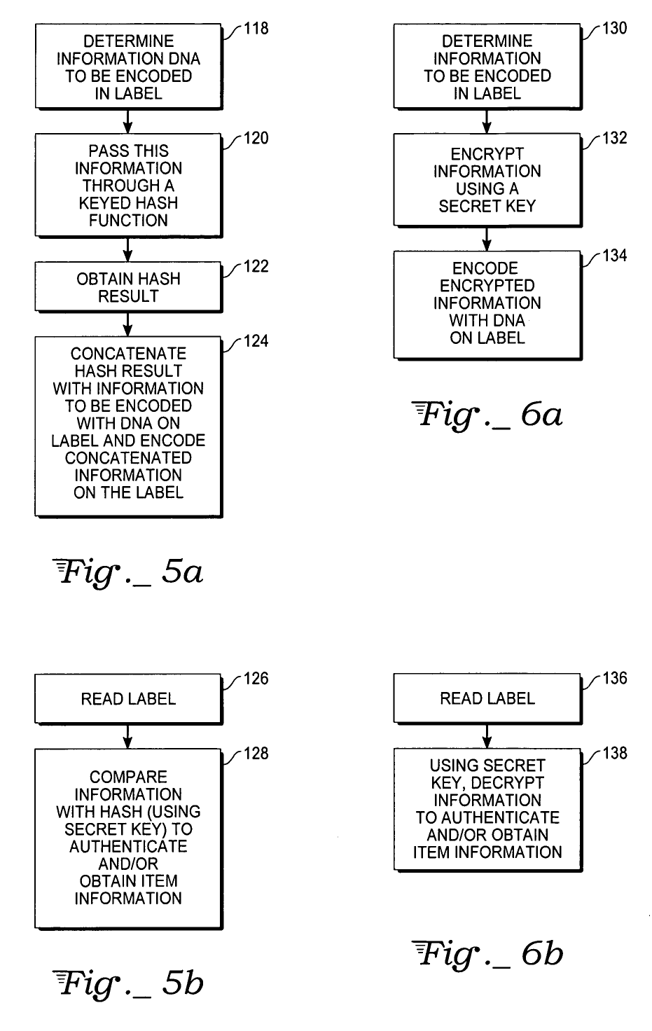

Information to be encoded in a label may be the result of a secure signature mechanism. With reference to FIG. 5a, the information to be encoded is identified (block 118) and then passed through a keyed hash function (block 120) to obtain a hash result (block 122). In one embodiment, the hash result is concatenated with the information to be encoded with DNA on the label; the concatenated information is then encoded on the label (block 124). In FIG. 5b, the label is read as described above (block 126). The information expected on the label is hashed with the known secret signature key and compared with the information obtained from the label in order to authenticate the item and/or obtain information about the item (block 128). This approach provides a reliable, non-malleable way to check the authenticity of the information contained in the label.

In another embodiment, a portion of or the entire hash result is encoded on the DNA label. The information in the DNA label is read and compared with that expected from the known secret signature key and the domain of the information that is encoded.

In yet another embodiment, the hash function may be unkeyed though a keyed hash function will provide greater security.

In another embodiment, shown in FIG. 6a, the information in the label may be encrypted. After determining what information is to be encoded in the label (block 130), the information is encrypted using a secret key (block 132). The encrypted information is then encoded with DNA on the label (block 134). In FIG. 6b, the label is read as described above (block 136). Using the secret key, the information is decrypted to authenticate and/or obtain the information about the item.

Error correction and detection techniques may be applied to the information encoded in the DNA label. Standard error correction and detection techniques, including interleaving and/or random re-orientation of the individual entities of the label (such as DNA segment choice, geometric location in an array), combined with the redundancies provided by error correction coding, significantly reduces the possibility of error in reading the DNA label. Error detection encoding can also be used to indicate when a reading error has occurred. Use of these techniques increases the robustness of the label and makes the label less susceptible to damage.

If security of the DNA label system described herein is compromised, a system may be put in place to quickly replace the secret keys and/or algorithms used to cryptographically sign and/or encrypt the information encoded in the label. By replacing the secret keys and/or algorithms quickly, the security level of the label system may be recovered quickly. After such a change has been made, compromised labels may be detected quickly, since these labels can be read only using the replaced key or decryption algorithm.

The techniques described above may be employed along with other product marking materials (such as barcodes (both 1-D and 2-D) and electronic product codes (“EPC”), including those used with radio frequency identification (“RFID”) transponders, etc.) which may be used to label the item with which the DNA label is or will be associated. The use of cryptographic techniques provides a mechanism to ascertain that the other product marking materials are bound to the DNA label. “EPC Tag Data Standards Version 1.1 Rev. 1.24,” Standard Specification, EPCglobal, 1 Apr. 2004 is hereby incorporated by reference.

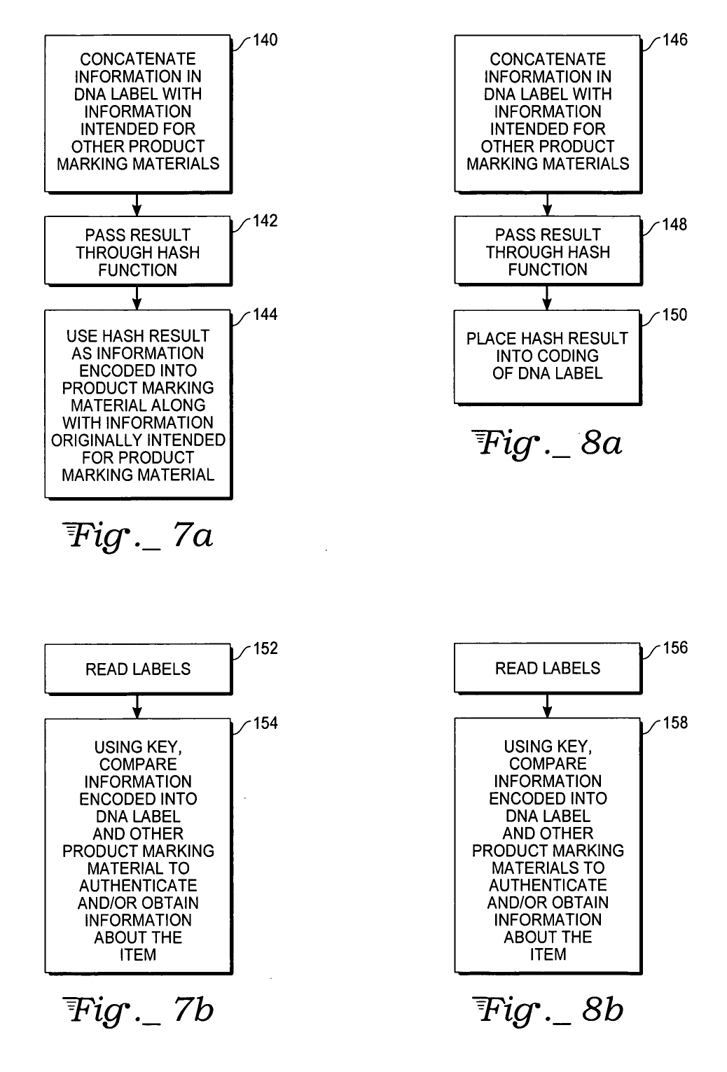

In FIG. 7a, the information to be encoded in the DNA label is concatenated with the information intended for the other product marking material(s) (e.g., serial number, manufacture code, date of manufacture, etc.) (block 140). The concatenated information is then passed through a hash function (which may be keyed or unkeyed) (block 142). A portion of or the entire hash result is then encoded into the product marking material along with the information originally intended for the product marking material before concatenation and hashing took place (block 144). In FIG. 7b, assuming a key is used, after both the DNA label and the other product marking material are read (block 152), the information encoded into the DNA label and the other product marking material are compared to authenticate and/or obtain the information about the item associated with the label (block 154).

In another embodiment, in FIG. 8a, the information to be encoded in the DNA label is concatenated with the information intended for the other product marking material(s). The result is passed through a hash function, which may be keyed or unkeyed (block 148). A portion or all of the hash result is encoded into the DNA label (block 150). In FIG. 8b, the information in both labels is read (block 156). Assuming a key is used, the information encoded into the DNA label is compared with the information encoded into the other product marking materials to authenticate and/or obtain the information about the item associated with the label (block 158).

In another embodiment, the DNA label may be encoded with or may be encoded to include single or multiple Uniform Resource Identifiers (“URIs”) that may be used as pointers to further information about the labeled items. For instance, the URI may point to a website storing information relevant to the item or part the label is associated with, including the product description, manufacturer, manufacture date, serial number, expiration date, etc. Other pointers may also be encoded into the label that are not pointers into the namespace of the WWW. The pointers provide a level of direction for access or reference to objects. RFC 3305 at http://www.itf.org/frc/rfc3305.txt?number=3305 discusses the relationship between URIs and URLs and also discusses namespaces and is hereby incorporated by reference. Secret keys may be used to authenticate and/or decode information accessed using the URI, demonstrating that the keys may be separated from actual labels but still used for authentication and/or decoding purposes. In another embodiment, a password may be required to access the information available using the URI.

The various approaches to authentication using DNA labels, either individually or in combination with other product marking materials, discussed above may be employed to authenticate items at discussed below.

DNA labels may be used to authenticate pharmaceuticals and medical devices. The DNA label (which may also be used in combination with some other type of product marking material) may be attached to packaging for pharmaceutical drugs to authenticate and/or obtain further information about the drugs. The DNA label may also be added to liquid medication, so the medication itself is “read” to authenticate the medication (the amount of DNA added in a label is small enough that no side effects will occur). The DNA label may be attached to an external or internal surface of a pharmaceutical capsule, or to the external surface of the “balls” of medicine contained in the pharmaceutical capsule. The DNA label, alone or in combination with some other product marking material, may also be attached to the packaging of or the body of a medical device, component of the medical device or spare parts of the medical device. The DNA label, alone or in combination with another product marking material, may be read to authenticate/and or obtain information about the pharmaceutical drugs and/or medical device, part, or spare part with which the DNA label is associated. If a DNA label is read and the item is not authenticated, the corresponding item may have been tampered with, may not be from an expected source, may be counterfeit, etc. If a DNA label is not present when one is expected, the associated item should clearly be viewed with suspicion.

Gaming machines and parts may also be authenticated with a DNA label (which may be used with or without another product marking material). For instance, a DNA label may be attached to a gaming machine (or components or spare parts of the gaming machine) such as a Pachinko machine, Pachislot machines, etc. The DNA label and any other product marking material used in conjunction with the DNA label can authenticate the machines and show that the machines have not been improperly modified. Other information, such as the last date of inspection, the date of manufacture, the serial number, etc. may also be carried in the DNA label or DNA label/product marking material combination. If no DNA label is present when one is expected, the item clearly cannot be authenticated.

Spare parts, such as those used to repair or modify cars, boats, motorcycles, aircraft, etc., may be authenticated with a DNA label (which may be used in conjunction with another product marking material). The DNA label and any associated product marking label may also be used as discussed above to convey further information about the item associated with the label.

DNA labels may also be employed in supply chain management schemes, for instance in a track and trace system. If DNA labels are attached or otherwise associated with parts, modules, and sub-assemblies, they can be used to verify the authenticity of the parts, etc., and their sources and may also be used to convey information about the parts, etc. Other product marking materials may be used in conjunction with the DNA labels.

DNA labels may also be used on pre-paid cards, such as phone cards. A DNA label or tag may be used to authenticate a card and indicate the monetary value of the card. The label or tag may also be used in combination with other recording techniques such as magnetic tape, electronic ship, smart card, or RFID. A change in the monetary value of the card can be updated on the DNA label or tag.

DNA labels may also be employed to authenticate and/or provide information about items other than those listed above. These include, but are not limited to: cosmetics; foodstuffs; hair shampoo; perfumes; ink-jet cartridges or toner cartridges for printers; electronics products, components, and circuits; batteries or cells; industrial raw materials; explosives; and other potentially contraband materials. A label may also be added to a message, document, or other communication. A label may be added to a message containing a key or keys that may be used to encrypt other messages, documents, communications, labels, etc.

In one embodiment of the invention, the DNA label may provide means for self-alignment of the DNA reader. An external indexing mechanism on the label will enable appropriate components of the reader to be properly aligned when reading the DNA label. One example of such an external indexing mechanism is a layout of reflectors or absorbers of radiant energy. The DNA reader could be configured to apply radiant energy to the label during the reading process, detect the reflection or absorption of radian energy, and adjust accordingly. Labels could also include a layout of marks that fluoresce when appropriately excited with radiant energy; these marks could be detected by a DNA reader which applied radiant energy to the label during the reading process and the appropriate label-reading components of the DNA reader could adjust themselves accordingly. Other examples include magnetic (micro)dots and marks consisting of a dye or dyes (printing inks, for example), that are photochromic and whose absorption spectra change after exposure to ultraviolet light.

What is claimed is:

1. A method for authenticating an item comprising:
a) using at least one known nucleotide sequence associated with a predetermined amount of information or at least one known non-natural nucleic acid analog sequence associated with a predetermined amount of information as a label to be associated with the item;
b) reading the label to detect the at least one known nucleotide sequence or the at least one known non-natural nucleic acid analog sequence;
c) authenticating the item if the read label contains the at least one identified nucleotide sequence or the at least one known non-natural nucleic acid analog sequence.

2. The method of claim 1 further comprising associating at least one known nucleotide sequence or at least one known non-natural nucleic acid analog sequence with the predetermined amount of information.

3. The method of claim 2 further comprising encoding information into the label using the least one known nucleotide sequence associated with the predetermined amount of information or the at least one known non-natural nucleic acid analog sequence associated with the predetermined amount of information, the encoded information including at least one of the following:
a) serial number;
b) part number;
c) manufacture code;
d) manufacture date; or
e) expiration date.

4. The method of claim 1 further comprising, after reading the label to detect the at least one nucleotide sequence or the at least known non-natural nucleic acid analog sequence, determining the information encoded into the label.

5. The method of claim 1 further comprising placing each of the at least one of a number of known nucleotide sequences or each of the least at one of a number of known non-natural nucleic acid analog sequences in at least one of a number of preidentified locations on the label.

6. The method of claim 5 wherein each of the at least one of the number of preidentified locations on the label is associated with a predetermined amount of information.

7. The method of claim 6 wherein the at least one known nucleotide sequence or the at least one known non-natural nucleic acid analog sequence located at the at least one of the number of preidentified locations on the label is associated with a predetermined amount of information.

8. The method of claim 3 further comprising passing the information to be encoded through a hash function, wherein the hash function is a keyed hash function or an unkeyed hash function.

9. The method of claim 8 further comprising concatenating a result of the hash function to the information encoded in the label and encoding at least part of the hash result into the label using at least one known nucleotide sequence or at least one known non-natural nucleic acid analog sequence.

10. The method of claim 9 further comprising determining the information encoded in the label, passing the information through a hash function, and comparing a result to the hash result encoded in the label.

11. The method of claim 3 further comprising encrypting the information to be included on the label and encoding the encrypted information in the label.

12. The method of claim 1 further comprising associating at least one other product marker with the item, wherein the other product marker includes at least one of the following:
a) a barcode;
b) an electronic product code; or
c) a radio frequency identification transponder.

13. The method of claim 12 wherein the at least one other product marker includes information about the item, the information including at least one of the following:
a) serial number;
b) part number;
c) manufacture code;
d) manufacture date;
e) expiration date.

14. The method of claim 13 further comprising concatenating information to be included in the label with information in the product marker and passing the concatenated information through a hash function and encoding at least part of a hash result into either the product marker or the label.

15. The method of claim 1 wherein the label is placed on either an external or an internal surface of the item.

16. A label for an item comprising information authenticating the item, wherein the information includes at least one known nucleotide sequence associated with a predetermined amount of data or at least one known non-natural nucleic acid analog sequence associated with a predetermined amount of data, either of the sequences detectable by a reagentless sensor, the predetermined amount of data authenticating or providing information about the item associated with the label.

17. The label of claim 16 further comprising information encoded into the at least one known nucleotide sequence or the at least one known non-natural nucleic acid analog sequence, the encoded information including at least one of the following:
a) serial number;
b) part number;
c) manufacture code;
d) manufacture date; or
e) expiration date.

18. The label of claim 16 further comprising each of the at least one nucleotide sequences or each of the at least one known non-natural nucleic acid analog sequence being located in at least one of a number of pre-identified locations on the label.

19. The label of claim 18 wherein each of the at least one of a number of pre-identified locations is associated with a predetermined amount of information.

20. The label of claim 17 wherein the encoded information has been passed through a hash function, wherein the hash function is a keyed hash function or an unkeyed hash function.

21. The label of claim 20 wherein a result of the hash function has been concatenated to the information encoded on the label, at least part of the result encoded into the label using at least one known nucleotide sequence or at least one known non-natural nucleic acid analog sequence.

22. The label of claim 17 further comprising at least one product marker is associated with the item, the at least one product marker including a least one of the following:
a) a barcode;
b) an electronic product code; or
c) a radio frequency identification transponder.

23. The label of claim 22 wherein the at least one product marker includes information about the item, the information including at least one of the following:
a) serial number;
b) part number;
c) manufacture code;
d) manufacture date; or
e) expiration date.

24. The label of claim 1 further comprising a means for self-alignment for a reader of the at least one known nucleotide sequence or the at least one known non-natural nucleic acid analog sequence.


SEQUENCE LISTING (2 sequences)

SEQ ID NO: 1 - DNA, 17 bp, Artificial Sequence
  Description of Artificial Sequence Synthetic polynucleotide sequence
actggccgtcgttttac

SEQ ID NO: 2 - DNA, 17 bp, Artificial Sequence
  Description of Artificial Sequence Synthetic polynucleotide sequence
cgtatcattggactggc